(19) United States
(12) Patent Application Publication
(10) Pub. No.: US 2026/0198869 A1
(43) Pub. Date: Jul. 16, 2026

(54) PATIENT RESTRAINT STRAP WITH MONITORING FUNCTION

(71) Applicant: TZU CHI UNIVERSITY, HUALIEN COUNTY (TW)

(72) Inventors: ZU-CHUN LIN, HUALIEN COUNTY (TW); TZU-YI YU, HUALIEN COUNTY (TW); JIA-HAO YANG, HUALIEN COUNTY (TW); CHIN-TING CHEN, HUALIEN COUNTY (TW)

(21) Appl. No.: 19/277,606

(22) Filed: Jul. 23, 2025

(30) Foreign Application Priority Data
Jan. 16, 2025 (TW) 114101890

(51) Int. Cl. A61B 5/00

(52) U.S. Cl. CPC A61B 5/746; A61B 5/6824; A61B 5/6831; A61B 5/702; A61B 2562/0247

(57) ABSTRACT

A patient restraint strap with a monitoring function includes a strap, having a first end and a second end; a first fastening unit for tightening the first end and second end, enabling the strap to generate a pressure value; a pressure detection unit for detecting the pressure value and giving a pressure warning signal when the pressure value is lower than a preset value; at least one position detection unit for detecting disconnection or displacement of the first fastening unit and giving a tightening warning signal when the first fastening unit is disconnected or displaced; a control unit, detachably connected to the strap, the control unit being in signal communication with the pressure detection unit and the position detection unit for receiving the pressure warning signal and/or the tightening warning signal and giving an alert and transmitting the alert to a remote device.

### FIELD OF THE INVENTION

The present invention relates to a patient restraint strap with a monitoring function, and more particularly, to a disposable or reuseable patient restraint strap which can be quickly wrapped around a patient's limb for monitoring the patient and has a timed reminder function.

### BACKGROUND OF THE INVENTION

In healthcare institutions, long-term care facilities, homes, etc., in order to prevent patients from engaging in harmful behaviors such as agitation, violence, removing needles/tubes involuntarily or self-harm, caregivers must use restraint devices such as restraint straps to restrict their movement. This is done to prevent patients from injuring themselves or others and to ensure the safety of both the patients and the surrounding caregivers.

Taiwan Utility Model Publication No. M537474 discloses a “restraint strap” published on March 1, 2017, comprising a binding unit, a fastening unit, a first connecting strap and a second connecting strap. The binding unit includes a binding member, a first fastener and a second fastener that can be interlocked with the first fastener. The fastening unit includes a third fastener disposed on the binding member and a fourth fastener that can be interlocked with the third fastener. The first connecting strap includes a first strap connected to one of the fastening unit and the binding member and a fifth fastener. The second connecting strap includes a second strap, a sixth fastener that is disposed on the second strap and detachably interlocked with the fifth fastener, and an outer ring. When in use, the sixth fastener passes through an outer perforation of the outer ring, the second strap and the outer ring form an outer loop with an adjustable diameter in a closed loop configuration. The outer loop can be secured on a fixed object, thereby achieving a stable restraint purpose.

Although the restraint strap as disclosed in the foregoing patent has the binding structure with multiple fasteners, it doesn’t provide any warning mechanism to alert caregivers if the patient breaks free after being restrained for a long time. It is potentially dangerous in use.

Taiwan Utility Model Publication No. M542454 discloses a “restraint device with a warning effect” published on June 1, 2017, comprising a restraint device, an elastic restraint strap, a first warning strap and a second warning strap. The restraint device is used to immobilize the patient. The elastic restraint strap is used together with the restraint device to immobilize the patient on the bed, chair or other auxiliary equipment. The elastic restraint strap is disposed in a certain position of the restraint device. Both ends of the elastic restraint strap are used for connection and fixing. After the restraint device immobilizes the patient, the elastic restraint strap provides the patient with a flexible and expandable range of movement. One non-retractable side of the first warning strap is fixed to one end of the elastic restraint strap, and the other side of the first warning strap has a detection warning element. One non-retractable side of the second warning strap is fixed to the other end of the elastic restraint strap, and the other side of the second warning strap has a detected unit. The detected unit and the detection warning element are couped in a magnetically attachable and detachable manner. While the restraint device immobilizes the patient, the elastic restraint strap provides the patient with sufficient space for movement. When the patient’s movement exceeds a preset range, the detection warning element of the first warning strap will be separated from the detected unit of the second warning strap, making a sound to warn the medical staff.

The detected unit and the detection warning element are coupled by means of magnetic attraction. A strong pull may cause them to detach from each other and trigger a warning, resulting in the drawback of false alarms.

Chinese Utility Model Registration No. CN203060006 discloses a “medical restraint pressure sensing alarm device” published on July 17, 2013, comprising a data acquisition module for collecting a numerical value; a control module for determining whether the numerical value reaches a threshold and sending an instruction; an alarm module for receiving the instruction from the control module and giving an alarm; and a power module for supplying power to the data acquisition module, the control module and the alarm module. The data acquisition module includes a restraint strap, a pressure sensor, an analog-to-digital converter, and a single-chip microcomputer. The restraint strap includes two elastic fabric layers and a plurality of pressure sensors between the two elastic fabric layers. When in use, after the patient is placed on the bed board, the restraint device immobilizes the patient and detects the intensity and frequency of the patient’s movement. The data acquisition module collects the patient’s movement data which is then transmitted to the control module for analysis and evaluation. Finally, the instruction is sent to the alarm module. Therefore, the device has the advantages of safe use, sensitive sensing, and flexible design. The device can detect patient discomfort in time, reduce the harm of unplanned extubation, and realize intelligent and personalized care for patients.

Although the device is equipped with the pressure sensor that detects changes in pressure to determine whether the patient has broken free from the restraint strap, the pressure sensor, the computing unit, the battery and other components are all integrated into the restraint strap and cannot be separated at will. As a result, the production cost of the restraint strap is relatively high, and it is difficult to clean and disinfect the restraint strap. Besides, it cannot be manufactured as a disposable or reusable restraint strap, nor can it give timed alerts (e.g., every two hours) to assist in loosening the restraints for the patient. It is still not ideal in use.

### SUMMARY OF THE INVENTION

In view of the shortcomings of the prior art, the primary object of the present invention is to provide a patient restraint strap with a monitoring function. The patient restraint strap comprises a strap, a first fastening unit, a pressure detection unit, at least one position detection unit, and a control unit. The strap has a first end and a second end. The first fastening unit is coupled to the strap for tightening the first end and second end, enabling the strap to generate a pressure value. The pressure detection unit is disposed on the strap. The pressure detection unit detects the pressure value and gives a pressure warning signal when the pressure value is lower than a preset value. The position detection unit is disposed on the first fastening unit. The position detection unit detects disconnection or displacement of the first fastening unit and gives a tightening warning signal when the first fastening unit is disconnected or displaced. The control unit is detachably connected to the strap. The control unit is in signal communication with the pressure detection unit and the position detection unit for receiving the pressure warning signal and/or the tightening warning signal and giving an alert.

Preferably, the first fastening unit includes a hook-and-loop strap and a fixing ring. The hook-and-loop strap is fixed to the first end. The fixing ring is fixed to the second end. When in use, the hook-and-loop strap passes through the fixing ring and is folded back and adhered to a back side of the hook-and-loop strap.

Preferably, the position detection unit is disposed between the hook-and-loop strap and the fixing ring. When the hook-and-loop strap and the fixing ring are disconnected, the position detection unit gives the tightening warning signal.

Preferably, the patient restraint strap further comprises a second fastening unit. The second fastening unit is coupled to the strap for tightening the strap. The position detection unit is disposed on the second fastening unit for detecting disconnection or displacement of the second fastening unit. When the second fastening unit is disconnected or displaced, the position detection unit gives the tightening warning signal.

Preferably, the second fastening unit includes two male fasteners and two female fasteners. The two male fasteners are fixed to the first end, respectively. The two female fasteners are fixed to the second end, respectively. The two male fasteners are engageable with the two female fasteners, respectively.

Preferably, the position detection unit is disposed between the two male fasteners and the two female fasteners. When any one of the two male fasteners and the two female fasteners is disconnected, the position detection unit gives the tightening warning signal.

Preferably, the two male fasteners each have an adjustment ring, allowing the two male fasteners to be adjusted in length.

Preferably, the two female fasteners each have a locking button. After the locking buttons of the two female fasteners are pressed, the two female fasteners are in a locked state to prevent accidental disengagement of the two male fasteners.

Alternatively, the second fastening unit includes a tightening knob, a drawstring, and a plurality of D-rings. The D-rings are fixed to a periphery of the strap. The drawstring passes through the D-rings in sequence. The drawstring has two ends connected to a circumference of the tightening knob. The tightening knob is fixed to the strap. When in use, the drawstring is tightened to secure the strap by rotating the tightening knob.

Preferably, a pad made of a skin-friendly material is provided on an inner side of the strap.

Preferably, the pressure detection unit is disposed on a surface of the pad or between the strap and the pad.

Preferably, two corresponding fixing belts are provided on the strap. A male buckle and a female buckle are provided on respective ends of the two fixing belts, enabling the fixing belts to be adjusted in length. When the male buckle and the female buckle are interlocked, the two fixing belts form a loop.

Preferably, when in use, the strap is tied on a patient’s limb to generate the pressure value. When the pressure value is lower than a preset value, the pressure detection unit gives the pressure warning signal. The preset value is the pressure exerted on the strap when the patient’s limb is about to break free from or loosen the strap.

Preferably, the position detection unit is a pressure sensor, a photoelectric sensor, an ultrasonic sensor, a magnetic sensor, a displacement sensor or a contact sensor.

Preferably, the control unit includes a computing module, a battery, a warning element, a wireless transmission module, a timer, a storage module, and an input interface. The battery, the warning element, the wireless transmission module, the timer, the storage module and the input interface are electrically connected to the computing module. The battery supplies power required for operation of the control unit.

Preferably, the control unit is detachably coupled to a retaining seat. The retaining seat is fixed to an outer side of the strap. The retaining seat is in signal connection with the pressure detection unit and the position detection unit for transmitting the pressure warning signal and the tightening warning signal.

Preferably, the alert is an audible signal or a light signal. The warning element is a buzzer or a warning light. After receiving the pressure warning signal or the tightening warning signal, the computing module controls the warning element to give a warning sound or a warning light.

Preferably, the wireless transmission module transmits the received pressure warning signal or tightening warning signal to a remote device. The remote device is a handheld electronic device, a tablet computer or a computer.

Preferably, the input interface is configured for inputting information including a patient’s name, bed number, restrained limb, and reminder time for release. The input information is stored in the storage module. The timer is configured for timekeeping. When the reminder time for release is reached, the warning element gives the alert and the wireless transmission module transmits the alert to a remote device.

According to the above technical features, the present invention has the following advantages:

1. The patient restraint strap provided by the present invention can detect whether the patient’s limb has broken free from the strap or whether the strap has been loosened on the patient’s limb, and it can immediately give an alert upon such an event to alert the caregiver or monitor to pay attention.

2. The dual-tightening mechanism of the first tightening unit and the second tightening unit can prevent the patient’s limb from breaking free or getting loose from the strap.

3. The control unit is designed as a detachable snap-fit mechanism, so it can be removed and separated from the strap at any time. Therefore, the control unit can be reused, and the production cost of the strap is low. Besides, the strap can be cleaned at any time, or it can be manufactured as a disposable strap to ensure cleanliness and hygiene during use.

4. The timer is used for timekeeping. When the reminder time for release is reached, the warning element will give an alert for reminding the caregiver or monitor to regularly release the patient’s restraint, allowing movement of the patient’s limb to prevent discomfort caused by prolonged restraint.

5. By means of pressure detection and disconnection detection and increasing the preset value of pressure detection, it is possible to determine whether the patient’s limb has broken free from the strap. It can timely detect that the patient’s limb is about to break free from or loosen the strap, so as to ensure the safety in use.

6. The strap has two open ends, defined as a first end and a second end, so that it can be quickly wrapped around the patient’s limb. The tightening knob can be operated easily to tighten the drawstring, which can increase the tightening force of the strap. Moreover, the strap can be tightened quickly in a very short time to reduce the chance that the patient’s limb breaks free from the strap in the wearing process.

### BRIEF DESCRIPTION OF THE DRAWINGS

FIG. 1 is a perspective view according to a first embodiment of the present invention, illustrating the inner side of the strap;

FIG. 2 is another perspective view according to the first embodiment of the present invention, illustrating the outer side of the strap;

FIG. 3 is a block diagram of the control unit according to the first embodiment of the present invention;

FIG. 4 is a schematic view according to the first embodiment of the present invention when in use, wherein the hook-and-loop strap passes through the fixing ring and is folded back;

FIG. 5 is a schematic view according to the first embodiment of the present invention when in use, wherein the male fastener is inserted in the female fastener to tighten the strap;

FIG. 6 is a schematic view according to the first embodiment of the present invention when in use, wherein the male buckle and the female buckle are interlocked for the fixing belts to be secured to the upright rod of the bed;

FIG. 7 is a schematic view according to the first embodiment of the present invention when in use, wherein the pressure detection unit detects the pressure value of the strap tied on the patient’s limb;

FIG. 8 is a schematic view of the second fastening unit according to a second embodiment of the present invention;

FIG. 9 is a schematic view according to the second embodiment of the present invention before the second tightening unit is tightened; and

FIG. 10 is a schematic view according to the second embodiment of the present invention after the second tightening unit is tightened.

### DETAILED DESCRIPTION OF THE PREFERRED EMBODIMENTS

Embodiments of the present invention will now be described, by way of example only, with reference to the accompanying drawings.

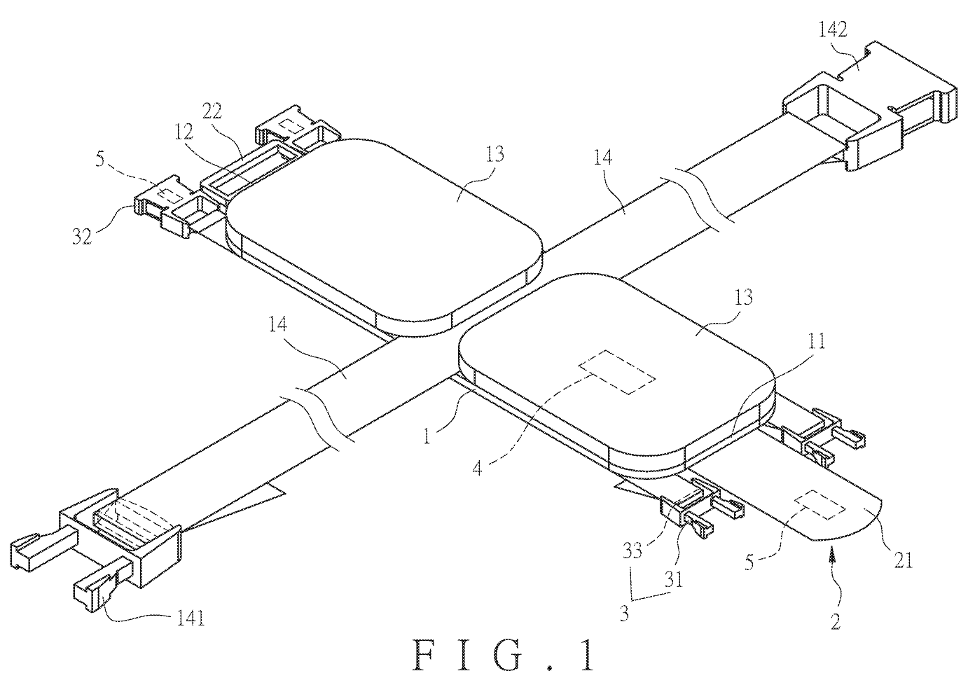

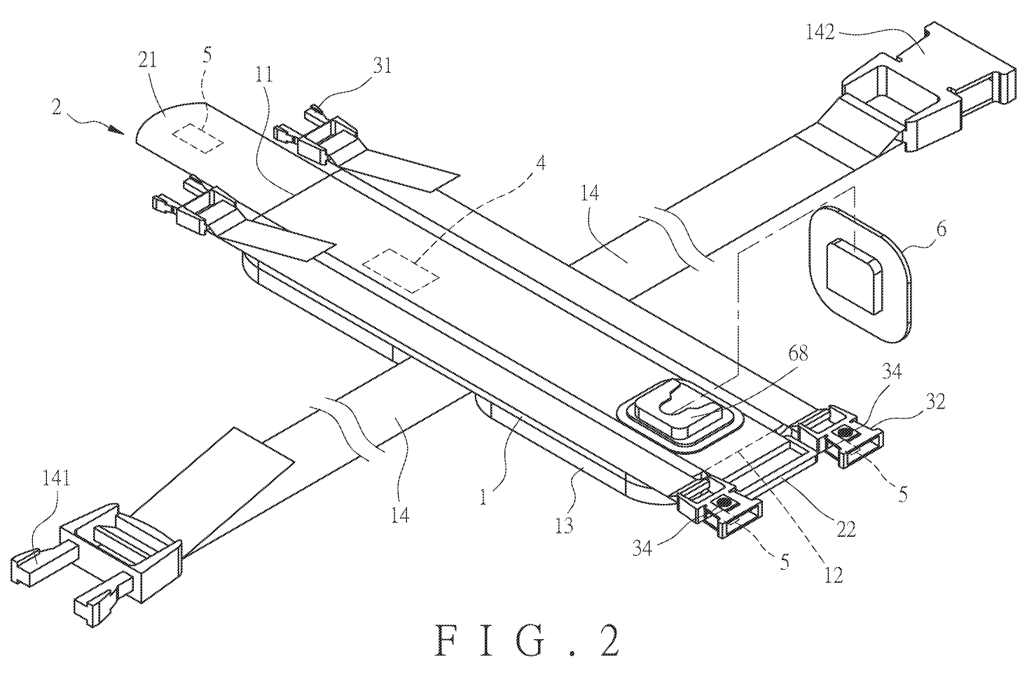

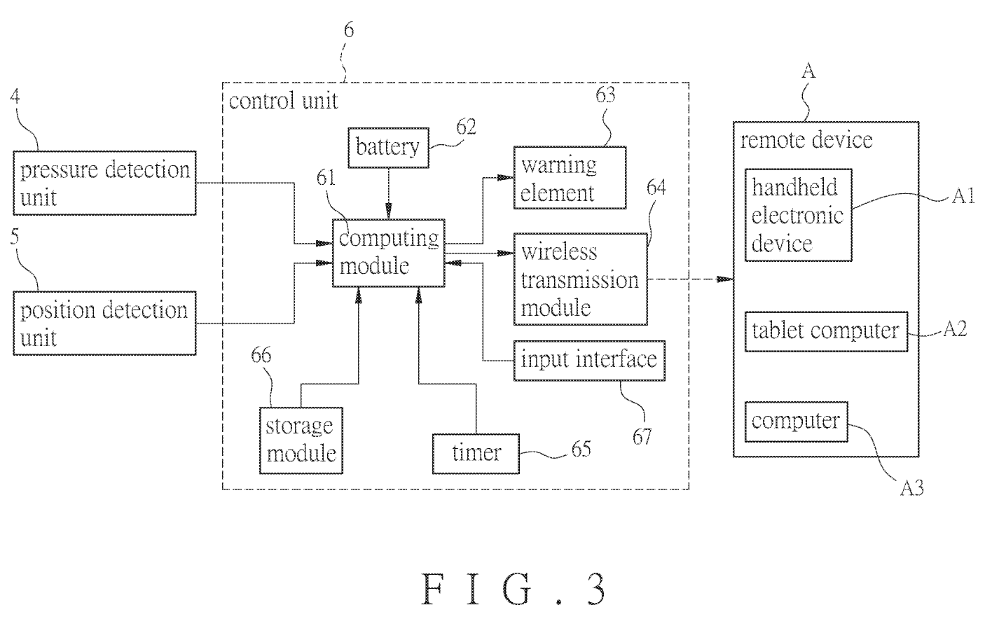

The present invention discloses a patient restraint strap with a monitoring function. Referring to FIG. 1, FIG. 2 and FIG. 3, a first embodiment of the present invention comprises a strap 1, a first fastening unit 2, a second fastening unit 3, a pressure detection unit 4, at least one position detection unit 5, and a control unit 6.

The strap 1 has two open ends, defined as a first end 11 and a second end 12. A pad 13 made of a skin-friendly material is provided on the inner side of the strap 1. Two corresponding fixing belts 14 are provided on the strap 1. A male buckle 141 and a female buckle 142 are provided on the respective ends of the two fixing belts 14, such that the fixing belts 14 are adjustable in length. When the male buckle 141 and the female buckle 142 are interlocked, the two fixing belts 14 form a loop, which can be secured to an upright rod of a bed frame with ease. The length of the fixing belt 14 having the male buckle 141 can be adjusted for controlling the range of movement of a patient.

The first fastening unit 2 is coupled to the strap 1. The first fastening unit 2 includes a hook-and-loop strap 21 and a fixing ring 22. The hook-and-loop strap 21 is fixed to the first end 11. The fixing ring 22 is fixed to the second end 12. When in use, the hook-and-loop strap 21 passes through the fixing ring 22 and is folded back and adhered to the back side of the hook-and-loop strap 21, thereby fastening and securing the strap 1 tightly around a patient’s limb.

The second fastening unit 3 is coupled to the strap 1. The second fastening unit 3 includes two male fasteners 31 and two female fasteners 32. The two male fasteners 31 are fixed to the first end 11, respectively. The two female fasteners 32 are fixed to the second end 12, respectively. The two male fasteners 31 are engageable with the two female fasteners 32, respectively. The two male fasteners 31 each have an adjustment ring 33, allowing the two male fasteners 31 to be adjusted in length, so as to fasten and secure the strap 1 tightly around the patient’s limb. The two female fasteners 32 each have a locking button 34. After the locking buttons 34 of the two female fasteners 32 are pressed, the two female fasteners 32 are in a locked state to prevent accidental disengagement of the two male fasteners 31.

The pressure detection unit 4 is disposed on the strap 1. The pressure detection unit 4 is disposed on the surface of the pad 13 or between the strap 1 and the pad 13 for detecting the pressure value of the strap 1 tied on the patient’s limb. When the pressure value is lower than a preset value, the pressure detection unit 4 will give a pressure warning signal. The preset value is the pressure exerted on the strap 1 when the patient’s limb is about to break free from or loosen the strap 1. When the pressure value is lower than the preset value, it can be considered that the patient’s limb has broken free from the strap or that the strap has been loosened on the patient’s limb. Thus, when the pressure value is lower than the preset value, the pressure detection unit will give a pressure warning signal as an alert. The preset value of the pressure detection can be increased, so that when the patient’s limb is not yet fully free but is about to break free from the strap, the decreasing pressure can be detected in advance. When there is a possibility of disengagement or loosening, a pressure warning signal can be given early, allowing timely or proactive detection and prevention of the strap 1 being broken free from or loosened.

The position detection unit 5 is disposed on the first fastening unit 2 and/or the second fastening unit 3 for detecting disconnection or displacement of the first fastening unit 2 and/or the second fastening unit 3. When the hook-and-loop strap 21 and the fixing ring 22 of the first fastening unit 2 are loosened, disconnected or displaced, the position detection unit 5 will give a tightening warning signal, and/or when any one of the two male fasteners 31 and the two female fasteners 32 of the second fastening unit 3 are loosened, disconnected or displaced, the position detection unit 5 will give the tightening warning signal. The position detection unit 5 may be a pressure sensor, a photoelectric sensor, an ultrasonic sensor, a magnetic sensor, a displacement sensor or a contact sensor. The first embodiment of the present invention has three position detection units 5 that are disposed between the hook-and-loop strap 21 and the fixing ring 22 as well as between the two male fasteners 31 and the two female fasteners 32, respectively. When there is disconnection or displacement between the hook-and-loop strap 21 and the fixing ring 22 or between the two male fasteners 31 and the two female fasteners 32, the position detection units 5 will give the tightening warning signal.

The control unit 6 is in signal communication with the pressure detection unit 4 and the position detection unit 5 for receiving the pressure warning signal and the tightening warning signal, thereby giving an alert and transmitting the alert to a remote device A. The control unit 6 includes a computing module 61, a battery 62, a warning element 63, a wireless transmission module 64, a timer 65, a storage module 66, and an input interface 67. The battery 62, the warning element 63, the wireless transmission module 64, the timer 65, the storage module 66 and the input interface 67 are electrically connected to the computing module 61. The control unit 6 is detachably coupled to a retaining seat 68. The retaining seat 68 is fixed to the outer side of the strap 1. The retaining seat 68 is connected to the pressure detection unit 4 and the position detection unit 5 in a wired manner. The battery 62 supplies the power required for operation of the control unit 6. The control unit 6 is detachably secured to the retaining seat 68, so that the computing module 61 can receive the pressure warning signal and the tightening warning signal through the retaining seat 68 and give the alert. The alert is an audible signal or a light signal. The warning element 63 may be a buzzer or a warning light. After receiving the pressure warning signal or the tightening warning signal, the computing module 61 controls the warning element 63 to give a warning sound or a warning light to alert the surrounding caregivers. The wireless transmission module 64 transmits the received pressure warning signal or tightening warning signal to the remote device A. The remote device A may be a handheld electronic device (mobile phone or smart watch) A1, a tablet computer A2 or a computer A3, so as to remind the remote monitor to pay attention. The input interface 67 is configured for inputting information such as the patient’s name, bed number, restrained limb, and reminder time for release. The input information is stored in the storage module 66. The timer 65 is configured for timekeeping. When the reminder time for release (for example, every 2 hours) is reached, the warning element 63 will give the alert. The wireless transmission module 64 transmits the alert to the remote device A for reminding the caregiver or monitor to regularly release the strap to relax the patient’s limb for massage and observation of blood circulation and assist in repositioning the patient to prevent injuries and discomfort caused by prolonged restraint.

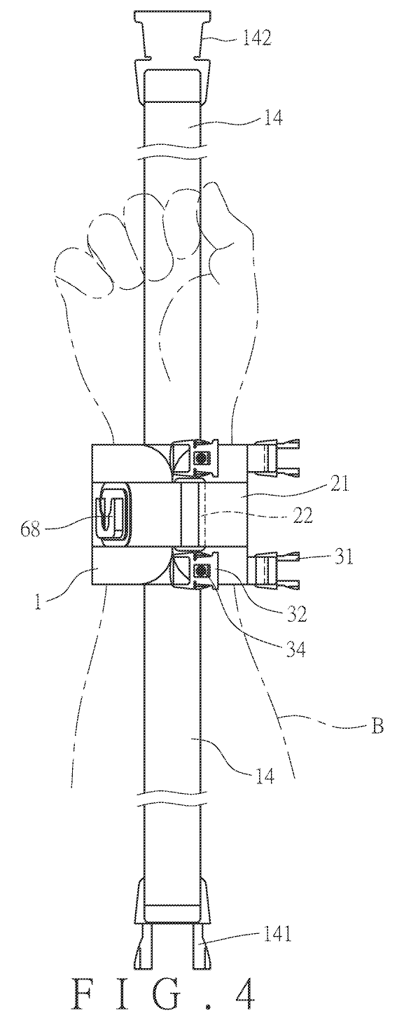

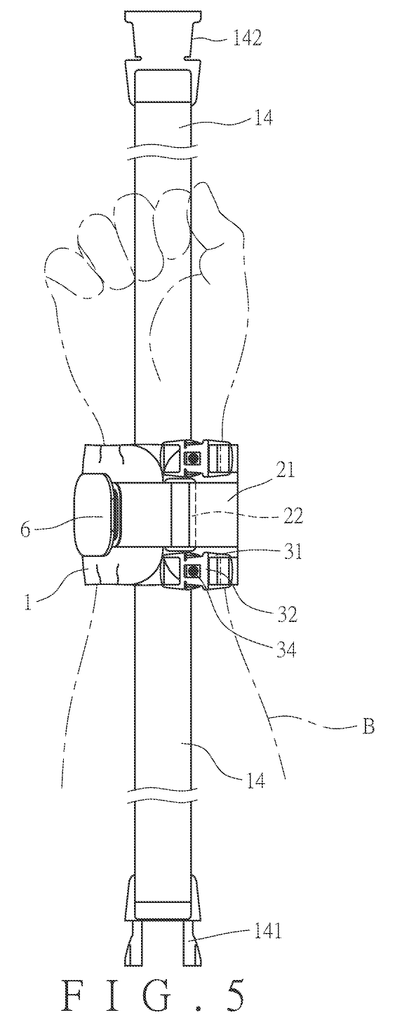

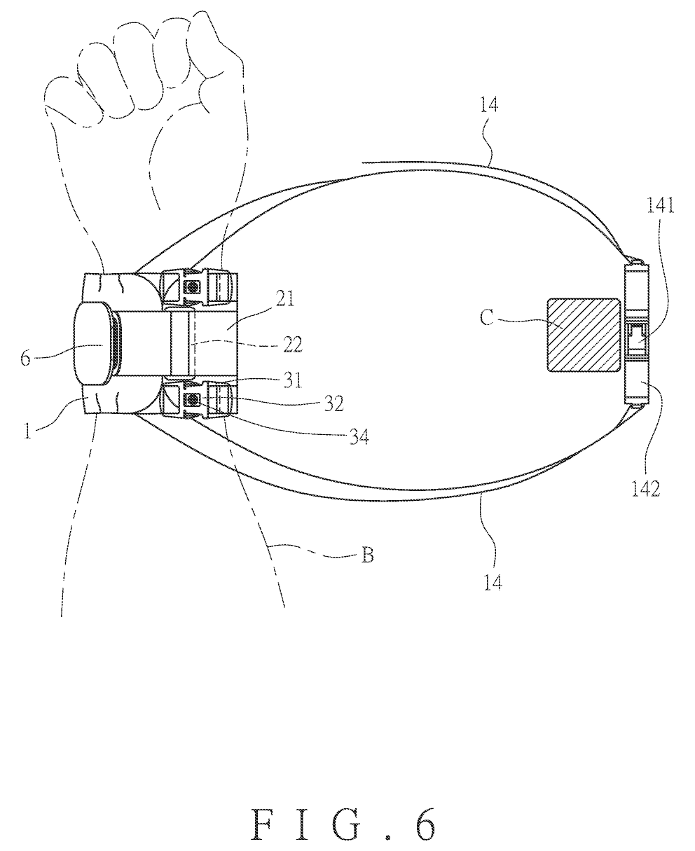

When in use, as shown in FIG. 1, FIG. 2 and FIG. 3, the strap 1 is wrapped around any one limb B of the patient’s limbs (as shown in FIG. 4), and the hook-and-loop strap 21 passes through the fixing ring 22 and is folded back and adhered to the back side of the hook-and-loop strap 21, thereby fastening and securing the strap 1 tightly around the patient’s limb B. After that, the two male fasteners 31 of the second fastening unit 3 are inserted in and secured to the two female fasteners 32, respectively. The adjustment rings 33 of the two male fasteners 31 are adjusted to tighten the lengths of the two male fasteners 31, such that the strap 1 is fastened and tightened on the patient’s limb B (as shown in FIG. 5). After the locking buttons 34 of the two female fasteners 32 are pressed, the two female fasteners 32 are in a locked state to prevent accidental disengagement of the two male fasteners 31. Then, the two fixing belts 14 on the strap 1 are fitted onto an upright rod C of a bed frame (as shown in FIG. 6). The male buckle 141 and the female buckle 142 are interlocked for the two fixing belts 14 to form a loop, which can be secured onto the upright rod C of the bed frame. The length of the fixing belt 14 having the male buckle 141 can be adjusted for controlling the range of movement of the patient.

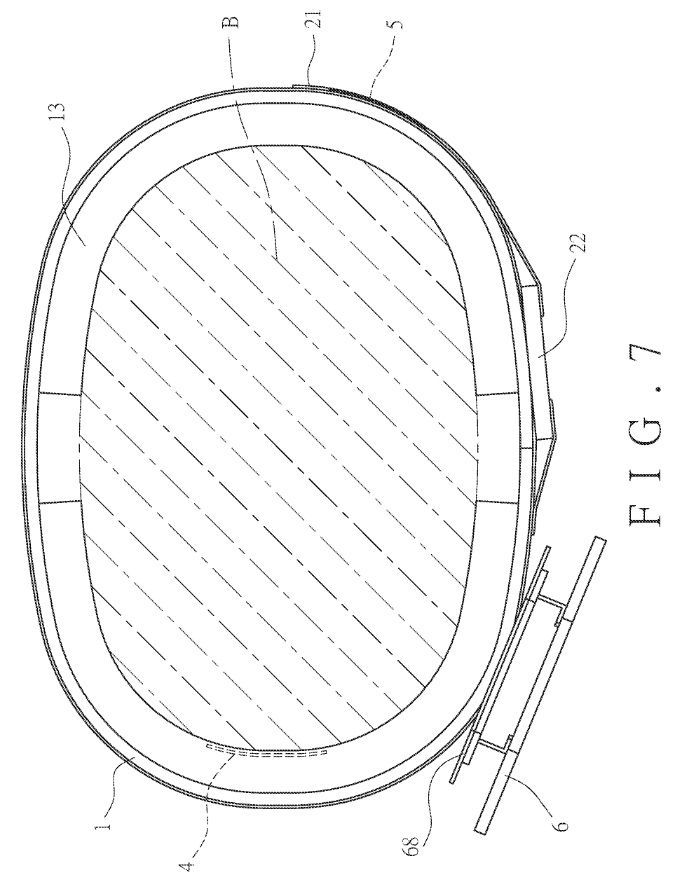

Finally, the control unit 6 is insertedly secured to the retaining seat 68. The battery 62 supplies power to start the pressure detection unit 4 for pressure detection and to start the position detection units 5 for disconnection or displacement detection. The pressure detection unit 4 detects the pressure value of the strap 1 tied on the patient’s limb B, as shown in FIG. 7. When the pressure value is lower than the preset value, the pressure detection unit 4 will give the pressure warning signal. Through the retaining seat 68, the computing module 61 receives the pressure warning signal. After receiving the pressure warning signal, the computing module 61 controls the warning element 63 to give a warning sound or a warning light to alert the surrounding caregivers. The wireless transmission module 64 transmits the received pressure warning signal to the remote device A, so as to remind the remote monitor to pay attention.

The position detection units 5 detect the disconnection or displacement of the first fastening unit 2 and/or the second fastening unit 3. When the hook-and-loop strap 21 and the fixing ring 22 of the first fastening unit 2 are loosened or disconnected, the corresponding position detection unit 5 will give the tightening warning signal, and/or when any one of the two male fasteners 31 and the two female fasteners 32 of the second fastening unit 3 are loosened, or disconnected, the corresponding position detection units 5 will give the tightening warning signal. Through the retaining seat 68, the computing module 61 receives the fastening warning signal. After receiving the fastening warning signal, the computing module 61 controls the warning element 63 to give a warning sound or a warning light to alert the surrounding caregivers. The wireless transmission module 64 transmits the received fastening warning signal to the remote device A, so as to remind the remote monitor to pay attention.

The timer 65 is used for timekeeping. When the reminder time for release is reached, the warning element 63 will give an alert. The wireless transmission module 64 transmits the alert to the remote device A for reminding the caregiver or monitor to regularly release the patient’s restraint, allowing limb movement to prevent stiffness and discomfort caused by prolonged restraint.

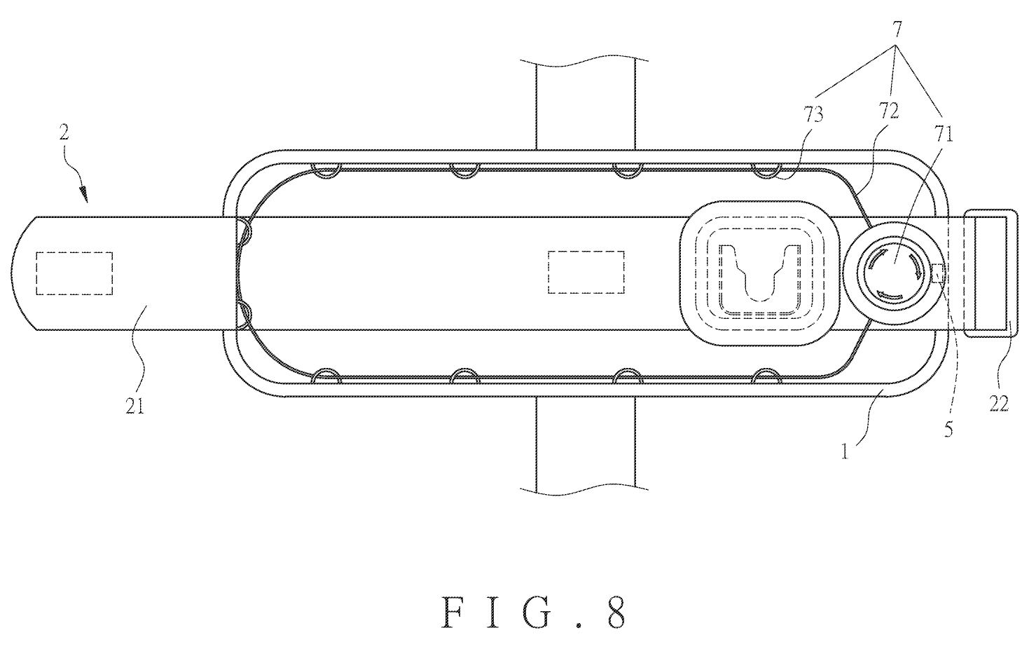

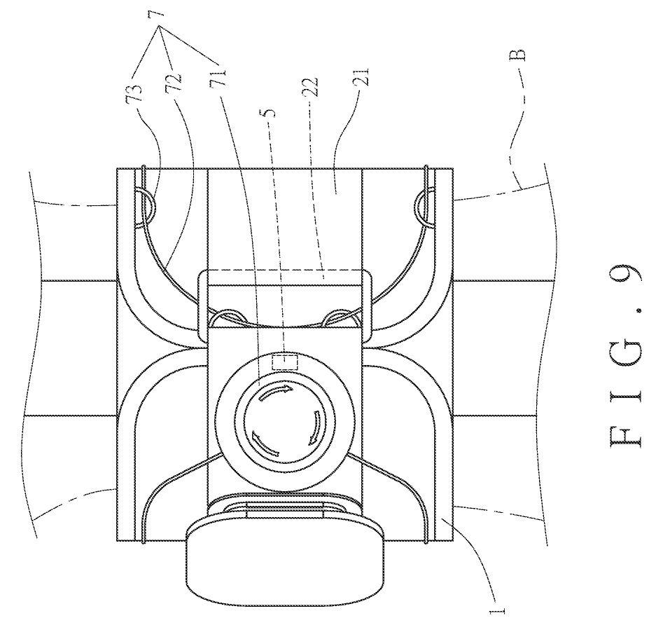

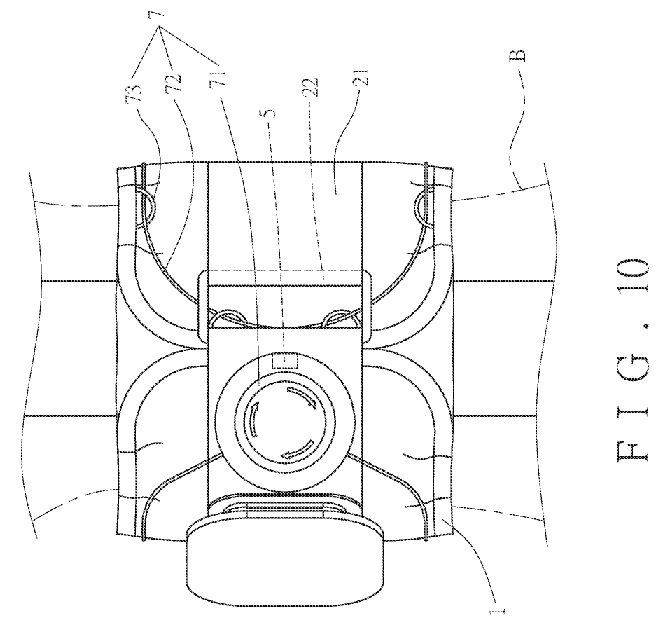

FIG. 8, FIG. 9 and FIG. 10 illustrate a second embodiment of the invention. The difference between the second embodiment and the first embodiment is the structure of the second fastening unit 7. The second fastening unit 7 is coupled to the strap 1. The second fastening unit 7 includes a tightening knob 71, a drawstring 72, and a plurality of D-rings 73. The D-rings 73 are fixed to the periphery of the strap 1. The drawstring 72 passes through the D-rings 73 in sequence. The drawstring 72 has two ends connected to the circumference of the tightening knob 71. The tightening knob 71 is fixed to the strap 1. By rotating the tightening knob 71, the drawstring 72 can be tightened to secure the strap 1. When in use, the strap 1 is wrapped around any one limb B of the patient’s limbs, and the hook-and-loop strap 21 passes through the fixing ring 22 and is folded back and adhered to the back side of the hook-and-loop strap 21, thereby fastening and securing the strap 1 tightly around the patient’s limb B. After that, the tightening knob 71 is rotated quickly to tighten the drawstring 72, which can increase the tightening force of the strap 1. Moreover, the strap can be tightened quickly in a very short time to reduce the chance that the patient’s limb breaks free from the strap in the wearing process. The position detection unit 5 may be a displacement sensor for detecting changes in the rotational position of the tightening knob 71 to determine whether the tightening knob 71 has been rotated and loosened and to immediately give the tightening warning signal, thereby preventing the patient’s limb B from being broken free or loosened from the strap 1.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A patient restraint strap with a monitoring function, comprising:
a strap, having a first end and a second end;
a first fastening unit, coupled to the strap for tightening the first end and second end, enabling the strap to generate a pressure value;
a pressure detection unit, disposed on the strap, the pressure detection unit detecting the pressure value and giving a pressure warning signal when the pressure value is lower than a preset value;
at least one position detection unit, disposed on the first fastening unit, the position detection unit detecting disconnection or displacement of the first fastening unit and giving a tightening warning signal when the first fastening unit is disconnected or displaced; and
a control unit, detachably connected to the strap, the control unit being in signal communication with the pressure detection unit and the position detection unit for receiving the pressure warning signal and/or the tightening warning signal and giving an alert.

2. The patient restraint strap as claimed in claim 1, wherein the first fastening unit includes a hook-and-loop strap and a fixing ring, the hook-and-loop strap is fixed to the first end, and the fixing ring is fixed to the second end, when in use, the hook-and-loop strap passes through the fixing ring and is folded back and adhered to a back side of the hook-and-loop strap.

3. The patient restraint strap as claimed in claim 2, wherein the position detection unit is disposed between the hook-and-loop strap and the fixing ring, when the hook-and-loop strap and the fixing ring are disconnected, the position detection unit gives the tightening warning signal.

4. The patient restraint strap as claimed in claim 1, further comprising a second fastening unit coupled to the strap for tightening the strap, wherein the position detection unit is disposed on the second fastening unit for detecting disconnection or displacement of the second fastening unit, when the second fastening unit is disconnected or displaced, the position detection unit gives the tightening warning signal.

5. The patient restraint strap as claimed in claim 4, wherein the second fastening unit includes two male fasteners and two female fasteners, the two male fasteners are fixed to the first end respectively, the two female fasteners are fixed to the second end respectively, and the two male fasteners are engageable with the two female fasteners, respectively.

6. The patient restraint strap as claimed in claim 5, wherein the position detection unit is disposed between the two male fasteners and the two female fasteners, when any one of the two male fasteners and the two female fasteners is disconnected, the position detection unit gives the tightening warning signal.

7. The patient restraint strap as claimed in claim 5, wherein the two male fasteners each have an adjustment ring, allowing the two male fasteners to be adjusted in length.

8. The patient restraint strap as claimed in claim 5, wherein the two female fasteners each have a locking button, after the locking buttons of the two female fasteners are pressed, the two female fasteners are in a locked state to prevent accidental disengagement of the two male fasteners.

9. The patient restraint strap as claimed in claim 4, wherein the second fastening unit includes a tightening knob, a drawstring and a plurality of D-rings, the D-rings are fixed to a periphery of the strap, the drawstring passes through the D-rings in sequence, the drawstring has two ends connected to a circumference of the tightening knob, and the tightening knob is fixed to the strap, when in use, the drawstring is tightened to secure the strap by rotating the tightening knob.

10. The patient restraint strap as claimed in claim 1, wherein a pad made of a skin-friendly material is provided on an inner side of the strap.

11. The patient restraint strap as claimed in claim 10, wherein the pressure detection unit is disposed on a surface of the pad or between the strap and the pad.

12. The patient restraint strap as claimed in claim 1, wherein two corresponding fixing belts are provided on the strap, a male buckle and a female buckle are provided on respective ends of the two fixing belts, enabling the fixing belts to be adjusted in length, when the male buckle and the female buckle are interlocked, the two fixing belts form a loop.

13. The patient restraint strap as claimed in claim 1, wherein when in use, the strap is tied on a patient’s limb to generate the pressure value, when the pressure value is lower than a preset value, the pressure detection unit gives the pressure warning signal, and the preset value is the pressure exerted on the strap when the patient’s limb is about to break free from or loosen the strap.

14. The patient restraint strap as claimed in claim 1, wherein the position detection unit is a pressure sensor, a photoelectric sensor, an ultrasonic sensor, a magnetic sensor, a displacement sensor or a contact sensor.

15. The patient restraint strap as claimed in claim 1, wherein the control unit includes a computing module, a battery, a warning element, a wireless transmission module, a timer, a storage module and an input interface, the battery, the warning element, the wireless transmission module, the timer, the storage module and the input interface are electrically connected to the computing module, and the battery supplies power required for operation of the control unit.

16. The patient restraint strap as claimed in claim 15, wherein the control unit is detachably coupled to a retaining seat, the retaining seat is fixed to an outer side of the strap, the retaining seat is in signal connection with the pressure detection unit and the position detection unit for transmitting the pressure warning signal and the tightening warning signal.

17. The patient restraint strap as claimed in claim 15, wherein the alert is an audible signal or a light signal, the warning element is a buzzer or a warning light, after receiving the pressure warning signal or the tightening warning signal, the computing module controls the warning element to give a warning sound or a warning light.

18. The patient restraint strap as claimed in claim 15, wherein the wireless transmission module transmits the received pressure warning signal or tightening warning signal to a remote device, and the remote device is a handheld electronic device, a tablet computer or a computer.

19. The patient restraint strap as claimed in claim 15, wherein the input interface is configured for inputting information including a patient’s name, bed number, restrained limb, and reminder time for release, the input information is stored in the storage module, and the timer is configured for timekeeping, when the reminder time for release is reached, the warning element gives the alert, and the wireless transmission module transmits the alert to a remote device.
